(19) United States
(12) Patent Application Publication
(10) Pub. No.: US 2025/0043343 A1
(43) Pub. Date: Feb. 6, 2025

(54) Spatially Mapped RNA Sequencing from Single Cells

(71) Applicant: The Board of Trustees of the University of Illinois, Urbana, IL (US)

(72) Inventors: Rashid BASHIR, Champaign, IL (US); Anurup GANGULI, Champaign, IL (US)

(21) Appl. No.: 17/440,746

(22) PCT Filed: Mar. 16, 2020
(86) PCT No.: PCT/US2020/022983

§ 371 Date: Sep. 17, 2021

Related U.S. Application Data
(60) Provisional application No. 62/820,578, filed on Mar. 19, 2019.

(51) Int. Cl. C12Q 1/6874; B01J 19/00

(52) U.S. Cl. CPC C12Q 1/6874; B01J 19/0046; B01J 2219/00317; B01J 2219/005; B01J 2219/00545; B01J 2219/00547; B01J 2219/00576

(57) ABSTRACT

Provided herein are methods of making a spatially-barcoded microarray, the method comprising the steps of: providing distinguishable mini-barcoded beads, wherein the distinguishable mini-barcoded beads comprise a plurality of distinct bead populations, with each bead member of a distinct population having an identical mini-barcode sequence; simultaneously delivering the plurality of distinguishable barcoded beads to a plurality of wells of a microarray, wherein a single bead is provided to each well; imaging the plurality of wells to identify the population type of each bead in each of the plurality of wells and thereby identify the mini-barcode in each well; removing the mini-barcodes from the beads and connecting the mini-barcode or a polymerase product of the mini-barcode to a surface of the well in which the bead is located; and removing the beads from the wells.

### CROSS-REFERENCE TO RELATED APPLICATIONS

This application is a U.S. National Stage Application filed under 35 U.S.C. § 371 of International Application No. PCT/US2020/022983, filed Mar. 16, 2020, which claims the benefit of U.S. Provisional Patent Application No. 62/820,578 filed Mar. 19, 2019, which are hereby incorporated by reference to the extent not inconsistent herewith.

### REFERENCE TO A SEQUENCE LISTING

A sequence listing containing SEQ ID NOs: 1-10 is provided herewith in a computer-readable form and incorporated by reference in its entirety and identified as follows: 2,379 bytes ASCII (Text) file named “69-18_US_SeqListing_ST25.txt,” created May 6, 2022.

### BACKGROUND OF INVENTION

Provided herein are methods for making spatially-barcoded microarrays, and related systems and applications that take advantage of the microarrays made by any of the disclosed methods.

There is a need in the art for spatial analysis of tissues and cells in order to assess heterogeneity in samples, including for understanding of biological state, disease diagnosis, and treatment efficacy, among others. Conventional techniques that offer spatial information from samples are low-throughput, laborious and time-consuming, with manual steps and poor signal-to-noise ratio. Generally, the conventional techniques rely on spot-application of nucleotide sequences (e.g., barcodes) on a substrate. The limit of reliable and consistent printing, is on the order of adjacent barcodes separated by about 100 μm or more. It is challenging to decrease this distance any further without suffering fundamental degradation associated with the reliable positioning of the printed material. Two barcodes that are 100 μm to 200 μm apart cannot achieve single cell resolution for cells that are smaller than 100 μm to 200 μm in size. Furthermore, the ability to achieve massive unique barcode sequences is challenging in the conventional linear spotting techniques.

For at least these reasons, there is a need in the art for improved microarrays having a spatial resolution that facilitates spatial detection at the cellular or sub-cellular level. In particular, current methods and devices that spot-print nucleotides in a microarray cannot reliably achieve such spatial resolution. The methods and systems provided herein address these problems in an efficient, reliable and easy to implement manner, such as by delivery of nucleotide sequences in a manner that achieves a high density (and corresponding cellular and even sub-cellular resolution), that reliably determines the spatial location of known nucleotide sequences, and that can provide a massive number of unique barcodes in a highly efficient manner.

### SUMMARY OF THE INVENTION

Provided herein are methods of making spatially-barcoded microarrays that achieve high spatial density by introducing to each well of a microarray a single bead having a minibarcode (MBC) affixed thereto. In this manner, the spatial resolution can be high, confined only in the size and spacing of the wells of the array, and corresponding well spatial density, to the corresponding beads. The methods rely on beads that are configured to be reliably imaged with a single bead positioned per well in a bead application step, with different bead populations distinguishable from each other. In this manner, the spatial resolution of the resultant system is not confined by the ability to reliably print known nucleotide sequences to specific locations. Instead, each bead population that together form the total beads applied, is uniquely identifiable, so that the corresponding specific MBC unique to a particular bead population is known in each well. The spatial pattern of MBC is correspondingly identified by the spatial pattern of beads over the microarray. The final barcode in each well is the sum of the sequential additions of beads to the microarray. In this manner, a vast number of unique barcodes (BC) are provided to the microarray, in a known spatial pattern due to the known sequential spatial pattern of beads applied to the microarray. The methods provided herein achieve significant time-savings because each bead application step is simultaneous and random, and is fundamentally different than the otherwise laborious sequential printing of unique barcodes over an array. The processes provided herein are exponential in nature, so that the number of unique barcodes becomes large very quickly, but in a reliably detectable manner.

Provided herein is a method of making a spatially-barcoded microarray, including by: providing distinguishable mini-barcoded beads, wherein the distinguishable mini-barcoded beads comprise a plurality of distinct bead populations, with each bead member of a distinct population having an identical mini-barcode sequence; simultaneously delivering the plurality of distinguishable barcoded beads to a plurality of wells of a microarray, wherein a single bead is provided to each well; imaging the plurality of wells to identify the population type of each bead in each of the plurality of wells and thereby identify the mini-barcode (MBC) in each well; removing the mini-barcodes from the beads and connecting the mini-barcode by a chemical, biochemical reaction such as ligation, polymerase extension, and the like, or a polymerase product of the mini-barcode to a surface of the well in which the bead is located; and removing the beads from the wells; thereby making a spatially-barcoded microarray.

In a most straightforward manner, the method effectively transfers the MBC from the bead surface to the well surface. Alternatively, methods may more broadly indirectly transfer a nucleotide sequence to the well surface by providing primers and/or probes that may be used to identify or amplify a target of interest in the well.

By sequentially repeating steps of delivering MBC-beads to wells, removing the mini-barcodes from the beads and ligating the mini-barcode and/or a polymerase product of the mini-barcode to a previously introduced mini-barcode in each of the plurality of wells, the resultant BC on the well surface is extended in length and variety to obtain a spatial pattern of barcode sequences over the plurality of wells; each barcode sequence comprising a plurality of known mini-barcode sequences and/or polymerase products from a plurality of mini-barcodes.

The sequentially repeating steps can be repeated any number of times, including to provide between two and ten repeats. In this manner the total number of unique BC's is defined by the formula (Bₚₒₚ)ⁿ, wherein n is the number of repeats and Bₚₒₚ is the number of uniquely identifiable bead populations and, accordingly, the number of unique MBC's in a beads introducing to the microarray step. The exponential nature of the process and, as discussed below, the ability to sub-divide introducing to the wells steps, provides a platform for rapidly increasing the total number of unique barcodes in the microarray.

The methods of making may further be described in terms of bead populations and delivery.

The bead in a well is optically, electrically, or mechanically identifiable by population type. The distinct bead populations may be identifiable by a bead color and/or a fluorescent label connected to the beads, the method further comprising the step of optically imaging the beads in the wells.

The step of simultaneously delivering the plurality of identifiable barcoded beads to the plurality of wells may comprise mixing the beads in a liquid and fluidically delivering the beads in the liquid to the plurality of wells, including by any of the means described in WO 2019/071142 filed Oct. 5, 2018 (Atty Ref. 336532: 91-17 WO), and which is specifically incorporated by reference herein.

The step of simultaneously delivering the plurality of identifiable barcoded beads to the plurality of wells comprises applying a physical force to insert the beads into the wells, including a centrifugal force and/or a magnetic force.

The plurality of bead populations may be formed from 1 or more than one distinct populations, including at least 5 distinct populations. One population may be used to effectively achieve “multiple” distinct populations by only loading fractions of the total number of wells in a sequential manner.

For example, when working with low number of bead populations e.g., 1 or 2, one can further divide a “Single bead loading step-n” into, for example, “x” smaller steps where in each step “x” only a fraction (e.g., 10%) of the array is filled. Then imaging after each step x confirms the well coordinates that are filled and empty and this allows one to track the new beads loaded in each step x. For each step x, we can deliver different MBC using the same bead population chronologically filling the entire well array and reaching 1 bead/well after the requisite number of steps (e.g., 10 for 10% wells filled per step). Once this state is reached, one can identify this as effectively corresponding to n=1 and then perform an enzymatic reaction. This way before the enzymatic reaction, the number of unique MBC on chip is much greater than the number of unique bead populations. In this manner, 1 bead loading step can be further broken into several smaller bead loading steps to achieve higher number of overall unique and identifiable MBCs on chip when working with, for example, only 1 unique bead population. The process will simply go faster if there is a higher number of bead populations to fill a higher fraction of the wells.

The plurality of bead populations may be applied to a fraction of the total number of wells of the microarray, such as between 5% and 50% of the total number of wells, and repeating the applying steps in one or more additional application steps to fill all wells of the microarray with a single bead, thereby effectively increasing the number of bead populations with unique mini-barcodes applied to the microarray.

The imaging step may comprise optically or electrically analyzing the microarray to identify the population type of each single bead in each well.

The removing the mini-barcode step may comprise cleaving the mini-barcode from the bead surface at a cleavage site, such as by photocleavage, chemical cleavage and/or temperature cleavage.

The connecting step may comprise ligating the mini-barcode to the well surface or to a mini-barcode previously connected to the well surface, such as by a 3′ to 5′ connection.

The connecting may comprise making an amplicon in the well by a polymerase reaction involving the mini-barcode, where the mini-barcode can act as a primer and attach to the preexisting barcode in the well. The polymerase will then elongate the pre-existing barcode in the well, thereby incorporating the reverse complement of the “new” MBC sequence introduced to the well. Similarly, a polymerase reaction may be performed on a MBC in the well and connecting the amplicon from the polymerase reaction to the well surface or to a previously connected mini-barcode. For any of the processes utilizing a polymerase reaction, the method further comprises the step of delivering reagents to the wells to perform the polymerase reaction in the well.

The methods provided herein are compatible with various sizes and ranges of relevant components. For example, the barcode connected to the well surface may comprise a plurality of mini-barcodes, the barcode having a nucleotide length that is greater than or equal to 20 bases, such as between 30 bases and 200 bases, and any sub-ranges thereof, such as between about 80 and 120 bases.

The mini-barcode may have a sequence length of about 4 nucleotides or greater, with respect to the portion of the MBC that will be the “active” portion of the barcode, or a has a sequence length of between 4 and 40 bases including other components, such as overlap region, oligodT portion, cleavable linker, T7 promotor, and other handle regions. Exemplary MBC lengths are between about 20 and 70 bases, with final barcode sequences having typical lengths of about 80 to 120 bases. The MBC for the first loading step may be generally longer, as it may include additional fixed portions, such as rna polymerase binding sites, etc., and for downstream library preparation. For example, MBC-bead loading steps 1-3 are delivered through beads, where the length of MBC1 is somewhat longer, about 50-70 bases, than MBC-bead loading steps 2-3, about 20-40 base length each (a portion of these MBC have the overlap region which will act as a primer and not become a part of the final barcode length).

The bead has a diameter selected to occupy greater than 50% of a well volume. Bead and corresponding well shape can also be custom designed to allow only 1 bead per well. The bead diameter may be greater than or equal to 200 nm and less than or equal to 5 mm. The methods provided herein are compatible with a range of bead sizes (and corresponding well sizes and spacing), depending on the application of interest. For example, the bead diameter can be sub-micron (e.g., as small as 200 nm) and can be as large as the application demands depending on the resolution (in the mm size regime, such as 1-5 mm). Generally, the criteria for lowest bead size includes: (1) The well volume or size should be about on the order of the same size as the bead volume or size. High resolution lithography techniques can yield sub-micron resolution well sizes, (2) Loading of beads into wells randomly via application forces, such as gravity, fluid (convective) forces, electrical, magnetic, electro-magnetic and/or centrifugation forces.

The wells of the microarray may be described as having a well density that is greater than or equal to 0.008 wells/μm² (8,000 wells/mm²) and/or a well spacing distance between adjacent wells that is less than or equal to 10 μm.

Such high spatial resolutions provide the ability to achieve a spatial resolution that is sub-cellular for intracellular characterization of a biological cell.

The wells of the microarray may have an average diameter that is greater than a bead diameter, such as no more than 50%, 30% or 10% greater than a bead diameter, thereby facilitating loading limit of one bead per well volume, including for beads spanning the range that is sub-micron (200 nm) to mm (e.g., 5 mm), and sub-ranges thereof.

Also provided herein are microarrays made by any of the methods described herein. As described, using the methods provided herein provides a reliable high-density microarray, with a unique barcode sequence known in each well of the microarray, including for sequencing a transcriptome from a biological material.

A high-density microarray may comprise: a microarray including a plurality of wells, each well having a known barcode sequence to form a spatially patterned array of nucleotide sequence barcodes, wherein the plurality of wells have a spatial density that is greater than or equal to 8,000 wells/mm² and a spatial resolution configured for intracellular or intercellular characterization of biological cells from a biological material.

The wells may have an average diameter of between 200 nm and 5 mm, such as between about 500 nm and 20 μm, with specific well sizes and, therefore, corresponding bead sizes depending on the application of interest and attendant desired resolution. Such resolution also constrains average separation distance between adjacent wells, such as a separation distance that is less than 10 μm, including between about 200 nm and 10 μm.

The barcodes may have an average nucleotide length that is greater than or equal to 20 bases, or greater than or equal to 100 bases, or between about 20 bases and 300 bases.

The wells may have a side wall configured to pixelate a biological tissue for spatial analysis of the biological tissue, such as for the reliable shearing of biological tissue that is forced into the wells. For example, there may be a relatively sharp edge between wells so that the tissue may be physically separated between adjacent wells.

Also provided herein are methods of using any of the microarrays of the present invention, including any microarray that, in turn, is made by a method described herein, including a high-throughput method of sequencing a transcriptome from a biological material.

The method may further comprise the steps of: overlaying the microarray with the biological material; pixelating the biological material into the plurality of wells; performing cDNA synthesis on the biological material in the plurality of wells, wherein non-barcoded random hexamers act as primers for the cDNA synthesis; ligating the barcoded sequences connected to the well surfaces that are complementary to the synthesized cDNA; and performing RNA sequencing on each of the plurality of wells.

The method may further comprise a pre-amplification step to amplify rare nucleic acid targets.

The method may be used to sequence a transcriptome at a sub-cellular spatial resolution. The sequenced transcriptome may be a complete transcriptome of a tissue or a cell.

The biological material may comprise a tissue sample or cells from a living subject.

The method may further comprise the step of delivering to the microwells microbeads having one or more reagents connected thereto, such as reagents comprising ligase enzymes and buffers lyophilized on a surface of the bead.

The delivering step may comprise magnetic pulldown of beads that are magnetic and/or centrifugation of beads dispersed in a carrier liquid.

The method may further comprise the step of providing reagents dispersed in a liquid to the wells of the microarray.

After performing cDNA synthesis, the synthesized cDNA may be dried in the microwells and a mixture of ligation and amplification reagents are provided to the microwells.

The lyophilized amplification reagents and enzymes may be delivered to the wells of the microarray after the ligation step.

The method may be for single cell or sub-cellular transcriptomics as the spatial density of the wells accommodates such high-resolution applications.

Any of the methods may have a single biological cell that spans a plurality of wells, optionally between 4 and 10 adjacent wells, thereby providing sub-cellular analysis.

Any of the methods may be described as providing a spatial resolution that is better than or equal to 20 μm.

Without wishing to be bound by any particular theory, there may be discussion herein of beliefs or understandings of underlying principles relating to the devices and methods disclosed herein. It is recognized that regardless of the ultimate correctness of any mechanistic explanation or hypothesis, an embodiment of the invention can nonetheless be operative and useful.

### BRIEF DESCRIPTION OF THE DRAWINGS

FIG. 1 is a flowchart summary schematic of a transcriptome analysis application with 5 steps, (i)-(v).

FIG. 2 is a schematic summary of sequential addition (four) of MBC-Beads to make a spatially-barcoded microarray.

FIG. 3 illustrates sequential addition of beads with MBC and corresponding transfer of MBC from the bead to the well surface, with a photocleavable (PC) linker that is cleaved to build-up the barcode.

FIG. 4 illustrates a bead in a well and corresponding transfer of MBC to the well surface by cleavage of the MBC portion from the bead in a first load (top portion) and a second load (bottom portion) to extend the MBC1 on the well surface to MBC1-MBC2.

FIG. 5 illustrates an example where the effective bead population number is effectively increased by introducing beads to a fraction of the microarray.

FIG. 6 is a schematic process diagram of measuring transcriptome from a tissue sample in one well of the microarray, with the seventh step having two versions.

FIG. 7 is an illustration of processing steps for the process of FIG. 9.

FIG. 8 is a barcode positional summary of the various components of the barcode that is connected to the well surface.

FIG. 9 summarizes use of the barcodes for RNA analysis of cells.

FIG. 10 is a literature summary of aspects relevant for barcoding design.

### DETAILED DESCRIPTION OF THE INVENTION

In the following description, numerous specific details of the devices, device components and methods of the present invention are set forth in order to provide a thorough explanation of the precise nature of the invention. It will be apparent, however, to those of skill in the art that the invention can be practiced without these specific details.

“Barcoded” refers to a nucleotide sequence connected to a well surface that can be used in an application to provide useful information about a biological material. “Spatially-barcoded” refers to a spatial distribution of barcodes, wherein at each position (e.g., each well) the barcode is known. In this manner, spatial information can be obtained when testing a biological material, including at the cellular or sub-cellular level.

“Distinguishable MBC beads” refers a bead mixture that comprises a plurality of distinct or different bead populations. Each bead population contains its own unique MBC sequence. Accordingly, by identifying the bead type, the MBC sequence is known. Accordingly, it is important that the MBC populations be identifiable from each other in an efficient, robust, and accurate manner.

In particular, the single beads in each of the wells of the microarray are broadly characterized as configured to undergo imaging. “Imaging” refers to identifying each of the plurality of bead population distributed over the microarray, and may be any of a number of imager types. For colored beads, the imaging may involve optical detection of bead color/fluorescence. Of course, the methods are compatible with any of a range of bead characteristics and combinations thereof, including size, shape, surface irregularities (e.g., optically-detectable surface features), radiological, electro-magnetic, mechanical property, density and the like.

“Pixelate” or “pixelating” refers to a tissue that has been subdivided into analyzable components, wherein the spatial location within the tissue is preserved. The pixelation may be any of the systems described in U.S. Pat. Pub. No. 2018/0119218, which is specifically incorporated by reference herein. For example, the tissue may be forced into the wells. The step of applying a force upon the microarray may be by any technique that reliably forces the deformable substrate into the plurality of wells, such that the tissue is sheared into separate pieces (e.g., “islands” or “pixelated”), with each piece in a unique well. Suitable force application techniques include by spinning the assembled microarray, tissue sample and deformable substrate in a centrifuge. The resultant centrifugal force accordingly forces the deformable substrate, and corresponding underlying tissue, into the wells. Similarly, a non-centrifugal uniform force may be applied over the deformable substrate, such as a weighted block or driver that results in desired deformable substrate deformation into the wells and corresponding shearing of the tissue sample into corresponding wells.

For the fluidic and bead delivery aspects to the microarray, any of the systems, components, and methods provided in WO 2019/071142 filed Oct. 5, 2018 (Atty Ref. 336532: 91-17 WO), which is specifically incorporated by reference herein, may be used.

Unless defined otherwise, “substantially” refers to a value that is within at least 20%, within at least 10%, or within at least 5% of a desired or true value. Substantially, accordingly, includes a value that matches a desired value.

The invention can be further understood by the following non-limiting examples.

### Example 1: Spatially Mapped RNA Sequencing from Single Cells from Tissue

Spatial analysis of tissues and cells offers important insights to heterogeneity in samples. Several techniques exist that can provide spatial information about the sample of interest such as LCM followed by qPCR, and fluorescent in situ hybridization (FISH).¹ However, those techniques suffer from low throughput, laborious and time-consuming manual steps, and poor signal-to-noise ratio.¹ Recently, Stahl et have demonstrated high throughput spatial mRNA sequencing on tissue,² where they captured mRNA on spatially barcoded oligodT probes followed by incorporation of this barcode into the tissue during the cDNA synthesis. Despite yielding uneven and low 5′ coverage of the mRNA transcript, barcoded oligodT probes have been preferred over random hexamers (an alternate priming strategy) in barcoding for two reasons. First, random hexamers contain 4096 independent sequences, and barcoding each is a time-consuming and expensive process. Second, barcoding random hexamers with another sequence significantly affects the binding of the hexamers to the transcript leading to poor cDNA yield.³ Another technique utilizing droplet-based single cell sequencing, also uses barcoded oligodT for cDNA synthesis.⁴ Like Stahl et al, that technique also suffers from low transcript coverage, and poor mRNA capture efficiency⁴.

Provided herein are two methods to sequence the complete transcriptome of tissues/cells at high throughput and at sub-cellular spatial resolution.

Method 1: Micro-well arrays are pre-spotted with barcoded oligodT probes. Tissue section is loaded and pixelated on the micro-well array as described in our earlier work.¹ Barcoded oligodT probes act as primers for cDNA synthesis. The cDNA synthesis will be followed by a pre-amplification step to enrich rare transcripts. Conventional off-chip RNA sequencing will be followed afterwards. The micro-wells provide a barrier for mRNA diffusion, and lower grid dimensions (spatial resolution that is as good as 1 μm) allows for sequencing of the complete transcriptome at the sub-cellular level.

Method 2: Non-barcoded random hexamers are used as primers for the RT step to produce cDNA. Non-barcoded random hexamer priming provides uniform coverage of the transcript with good cDNA yield compared to oligodT priming. This allows the cDNA synthesis to be performed in the “natural” or closest to standard off-chip state. Barcodes are spatially pre-spotted in the micro-wells (unique spatially barcoded sequence containing the coordinate data in each well), and are ligated to the cDNA after the natural cDNA synthesis. This can be followed by a spatially conserved pre-amplification step in each well to enrich rare nucleic acid targets at any coordinate/well. Conventional off-chip RNA sequencing will be followed afterwards.

Two strategies may be utilized: (i) In a first strategy, ligase enzymes and buffers are lyophilized on beads, and delivered to micro-wells using magnetic pulldown (magnetic beads) or centrifugation after dispersion of the beads in an immiscible carrier liquid (carrier liquid will float on top of the wells due to lower density than water based reagents in wells). This immiscible carrier liquid will be less dense than the aqueous reagents inside wells, and will prevent cross-talk between any two adjacent wells. Other methods of lyophilized or dried ligation reagents can also be used; (ii) In a second strategy, we dry out the cDNA and reagents used for reverse-transcription post the reverse transcription step. A cocktail containing both the ligation and amplification reagents are loaded into the wells as described in our previous work. Ligation reaction takes place first, followed by amplification based on reaction temperature. In another version, the lyophilized amplification reagents and enzymes are delivered to the well array post ligation step.

The above methods can be extended to work with cells to achieve single cell or sub-cellular transcriptomics, where, instead of loading and pixelating tissue sections, we capture the cells on our pre-spotted spatially barcoded micro-well array. The downstream processes are the same as outlined in the Methods above. The identity of the cellular/sub-cellular transcriptome is tracked using the cells spatial location after capture on the microwell array, as the spatial pattern of barcodes on the micro-well array is known (see, e.g., Example 2).

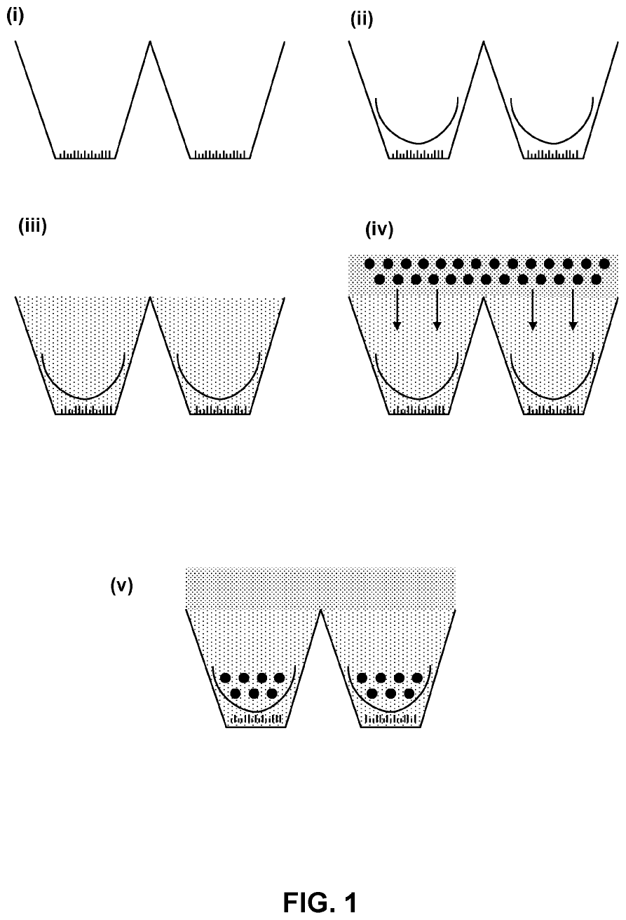

FIG. 1 is a schematic summary of a method of determining the transcriptome from a tissue using any of the microarrays (two wells illustrated) made by any of the methods described herein. In panel (i), the microwell array is provided, wherein each well has a unique (and known) nucleic acid barcode or sequence. The microwell array is effectively “prespotted” with unique nucleic acid barcode in each well. In panel (ii), the tissue is pixelated, so that sample is provided in each well. Any number of processing steps may be performed on the tissue depending on the application of interest. For example, as desired, tissue fixation and permeabilization can be performed on the pixelated tissue, including before or after pixilation. Similarly, the RNA can be chemically or physically fragmented into smaller sizes before cDNA synthesis. In panel (iii), cDNA synthesis is performed in each well, including using a kit with random hexamer added. Panel (iv) illustrates a carrier fluid with beads provided to or in the carrier fluid, that can be forced into the microwells, such as by centrifugation and/or magnetic bead pull down. The beads may carry any number or types of materials, including reagents, enzymes and the like, required for the particular application. Ligase enzymes and reagents lyophilized on beads added in an immiscible carrier fluid are one means for introducing appropriate material to the microwells. The step summarized in Panel (iv) can be replaced by drying of formed cDNA in each well and adding of a second solution with ligation reagents. Panel (v) illustrates ligation of the cDNA to the corresponding complementary barcodes attached to the well surface. After ligation of barcodes in the step shown in Panel (v), a spatially conserved (in each well) on chip amplification can be performed to increase the copy numbers of rare transcripts. Amplification can be linear (RNA polymerase) or exponential (DNA polymerase).

The barcodes in each well will have a position specific sequence, an illumine identifier sequence (or similar), sequence for on chip and off-chip amplification primers.

Enzymes for on chip amplification can be already present at the step shown in Panel (iii) or added after the step shown in Panel (v) using enzyme lyophilized or dried on beads similar to step (iv).

### EXAMPLE 1 REFERENCES

1 A. Ganguli, A. Ornob, N. Spegazzini, Y. Liu, G. Damhorst, T. Ghonge, B. Thornton, C. J. Konopka, W. Dobrucki, S. E. Clare, R. Bhargava, A. M. Smith, F. Kosari and R. Bashir, Nat. Commun., 2018, 9, 202.
2 P. L. Stahl, F. Salmen, S. Vickovic, A. Lundmark, J. F. Navarro, J. Magnusson, S. Giacomello, M. Asp, J. O. Westholm, M. Huss, A. Mollbrink, S. Linnarsson, S. Codeluppi, A. Borg, F. Ponten, P. I. Costea, P. Sahlen, J. Mulder, O. Bergmann, J. Lundeberg and J. Frisen, Science (80-.)., 2016, 353, 78-82.
3 J. H. Lee, E. R. Daugharthy, J. Scheiman, R. Kalhor, R. Amamoto, D. T. Peters, B. M. Turczyk, A. H. Marblestone, J. L. Yang, T. C. Ferrante, R. Terry, S. S. F. Jeanty, C. Li, R. Amamoto, D. T. Peters, B. M. Turczyk, A. H. Marblestone, S. a Inverso, A. Bernard, P. Mali, X. Rios, J. Aach and G. M. Church, Science, 2014, 343, 1360-1363.
4 A. M. Klein, L. Mazutis, I. Akartuna, N. Tallapragada, A. Veres, V. Li, L. Peshkin, D. A. Weitz and M. W. Kirschner, Cell. May, 2015, 21, 1187-1201.

### Example 2: Methods of Making the Microarray

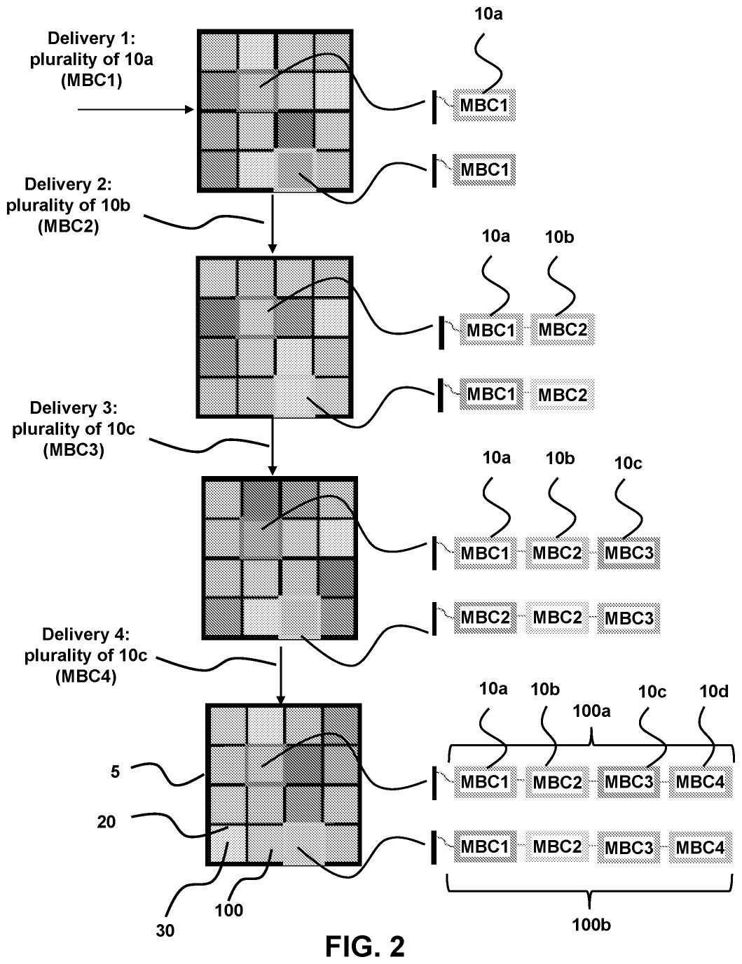

FIG. 2 illustrates a method of making a spatially-barcoded microarray 5. A mini-barcode (MBC) 10a is delivered to the microarray 20 having a plurality of wells 30. The barcodes 100a 100b are connected to a surface of a well 30. Two exemplary barcodes 100a and 100b are illustrated for four sequential deliveries of mini-barcodes (MBC) 10 to the microarray, illustrated as 10a, 10b, 10c, and 10d, respectively and corresponding MBC1-MBC4 provided in deliveries 1-4. By selecting the volume and size of the well to the bead size to ensure only one bead is delivered to one well, the system is configured to reliably detect the exact sequence delivered to each well by detecting the bead type in the well. For the illustrated microarray 20 having a 4×4 array of wells 30, focus is provided on well position (2, 2) and (3, 4). Ahead of the delivery, provided are distinguishable mini-barcoded beads that comprise a plurality of distinct bead populations. In the example of FIG. 2, the populations are distinguished by color, such as bead color (including by a fluorescent label connected to the bead). For a given bead population, the sequence MBC is known for that population. For mini-barcoded beads having, for example, an eight distinct (distinguishable) populations, for each delivery to the 4×4 array, there will be some duplicates. For four sequential deliveries, where between deliveries the MBC is released from the bead and the beads removed and the MBC connected in the well (to the well surface for the first delivery, then to an already-present MBC sequence extending from the well surface), there are a possible 84 (4096) unique barcodes in the well. The barcodes 100a and 100b are illustrated for wells (2, 2) and (3, 4), respectively, where unique MBC colors indicate unique sequences. That is, MBC1 is one of eight unique sequences, similarly for each of MBC2-MBC4, for 84 possible unique sequences.

On-chip synthesis of the spatial barcode array in FIG. 2 is illustrated by optical imaging of the beads, so that each specific bead in each well is distinguished and identified. For other bead systems having different distinguishing characteristics, the imaging may reflect other non-colorimetric characteristics, such as radiological, mechanical or size. For the color-based system, MBC in each well is known by identifying bead color in each well. Biochemical joining/extension of the MBC may be facilitated using polymerase/ligase enzymes. The color spatial pattern is formed due to the different/unique permutation and combinations of the MBC's randomly delivered to each well. To track what MBC is provided to each well, the unique bead type/population (e.g. color or fluorescence) to which the MBC is attached is identified by optically imaging in between each addition/elongation step. In this manner, the practical limit to spatial resolution of the microarray is the size of the bead that can be reliably imaged, as the wells are configured to have a volume/shape such that a single bead is provided to each well, or remains after a processing step, such as rinsing to flush out a “stacked” bead in a well.

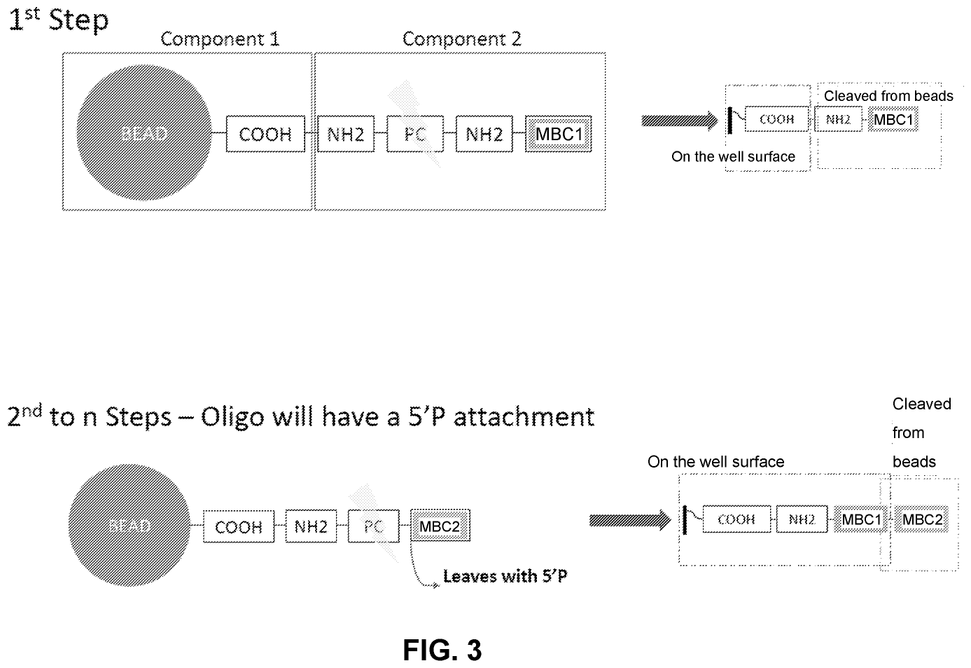

FIG. 3 illustrates a bead with MBC sequence connected thereto on the left side (Component 1 and Component 2). In the first step (e.g., the first delivery step of FIG. 2) the MBC may be connected to the bead via a linker to facilitate linking to the well surface. For example, via a (well surface)-COOH—NH₂-MBC linkage. A cleavage site may be provided between the bead and the MBC sequence, including a photocleavable site (PC). In this manner, each bead that is positioned in a single well, may cleave the MBC and linkage sequence to provide MBC1 to the well surface. As the beads are all identifiable in the wells, and each bead population has a known MBC1 sequence, with different populations having different MBC1 sequence (MBC1_{red}; MBC1_{green}; MBC1_{blue}; etc.), the spatial map of colors provides a spatial map of MBC sequences, so that each MBC in each well is known. After the MBC is reliably connected, the beads may be removed, such as by magnets, centrifugation/gravity, and/or rinsing, wherein the adhesive force (e.g., chemical bond) between the well surface and MBC is greater than the bead removal force. Subsequent steps repeat the first step, although with some variation in the how the MBC(2 . . . n), where n is the total number of steps or repeats, is provided to the previously provided MBC, e.g., MBC(1 . . . (n−1)), such as 5′ to 3′ connection, including by a ligation mechanism. In this manner the barcode in each well is sequentially built up by sequencing addition of MBCₓ. where x ranges from 1 to n.

Component 1 contains a bead. The bead surface can be functionalized for attachment of MBC, including with a carboxyl group that is compatible with further functionalization. Exemplary beads include those by Spherotech/Luminex, having about a 5 μm size and COOH modified. The system is compatible with any number of bead populations and types, such as about 10 fluorescently-distinguishable colors, up to 100 or more optically unique beads, based on one or a combination of colors, shapes, surface roughness or irregularity or the like. Component 2 contains the desired MBC and a cleavage region, including a chemical or photocleavable cleavage region (PC). Amine groups may be used to facilitate linkage to carboxyl groups connected to the bead and to the PC region. The right side of FIG. 3 indicates the sequence that is connected to the well surface from the first step (carboxyl-amine covalent bonding between well surface and MBC1 sequence) and for subsequent sequential addition of MBC2 . . . MBCn, over n sequential additional steps where the subsequent MBCx may be ligated to previously provided MBC(x−1), such as via an oligonucleotide having a 5′ P attachment.

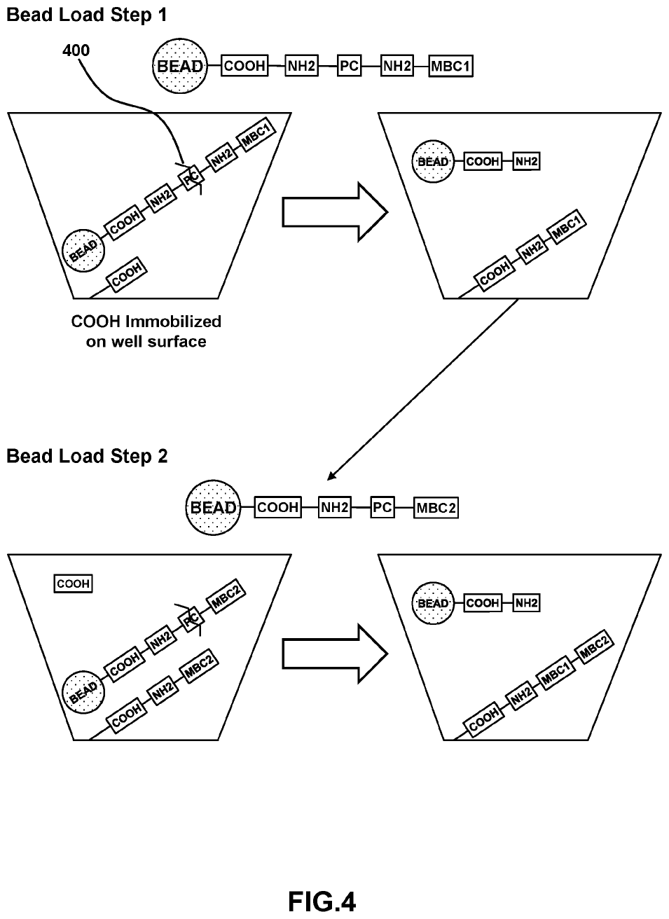

FIG. 4 is a further detailed schematic summary of the addition of MBC from a bead to a well surface. The systems and methods provided herein ensure a single bead is provided to each well of interest, with each bead in each well having an identifiable characteristic so that the MBC sequence provided to each well is known. MBC is released from the bead, as indicated by the PC component cleaved as indicated by 400 (e.g., photocleaved) making MBC1 available to bond to the well surface, including a functionalized well surface to mediate bonding between a linker portion connected to the MBC molecule (e.g., NH₂) and the well surface (e.g., COOH functionalized surface). The bead is removed from the well and a subsequent bead with MBC2 connected to the bead surface introduced to the well (“Bead Load Step 2” shown in the bottom panels). The plurality of bead populations in subsequent sequential steps may be configured to facilitate directed 5′ to 3′ ligation between MBCx and MBCx-1, wherein 2≤x≤n, with n being the total number of additions.

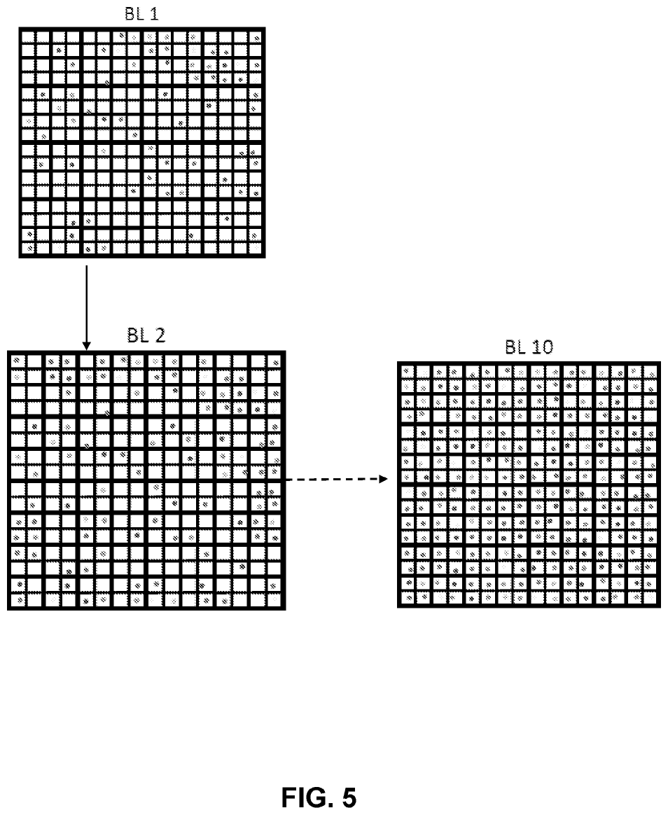

In this manner, a very large number of unique barcodes are spatially arranged in a microarray, according to the formula (Bₚₒₚ)ⁿ, wherein Bₚₒₚ is the number of distinguishable bead populations and n is the number of sequential additions. For example, for Bₚₒₚ=10 (e.g., 10 uniquely identifiable bead colors, each color having a known MBC) and 4 sequential additions, the total number of possible unique barcodes generated after the fourth sequential random addition is 104 or 10,000 barcodes. FIG. 5 is intended to illustrate the flexibility of the process in the event a higher Bₚₒₚ is desired but it is not feasible to distinguish such a high number for Bₚₒₚ. Instead, each sequential step can be “sub-divided” into multiple steps, such that for sub-step only a fraction of the wells are filled. So for Bₚₒₚ=10, but there is a desired Bₚₒₚ of 100, each sub-step may fill only 1/10 of the wells with a bead, followed by another 1/10 introduction but where each of the “colors” have a different sequence, in this manner an effective Bₚₒₚ of 100 is achieved, although there may only be 10 optically distinguishable populations, thereby providing 1004 barcodes rather than 104. Accordingly, any of the methods and systems provided herein may be described in terms of achieving a total number of barcodes over a certain number of sequential steps, sub-steps within the steps, and corresponding fraction of the wells filled with a bead during each sub-step.

The methods and systems are compatible with a range of well fractions being filled, including between 0.05 (20 sub-steps) and 0.5 (2 sub-steps), and any subranges thereof. Microarray covers may be provided that make any desired fraction of the wells of the microarray inaccessible to beads. In this manner, for any bead-loading (BL) step, the effective Bₚₒₚ may be increased. If a total of 100 MBC types is desired but only 10 bead populations are available, each BL step is repeated such that only 1/10 of the array is filled per BL step, and that is repeated 10 times with each repeat having a different sequence for each of the populations. The wells can be imaged to confirm that all wells are filled and MBC added to the well surface, or a polymerase extension may be performed.

### Example 3: Single Cell Resolution for Spatial Transcriptomics

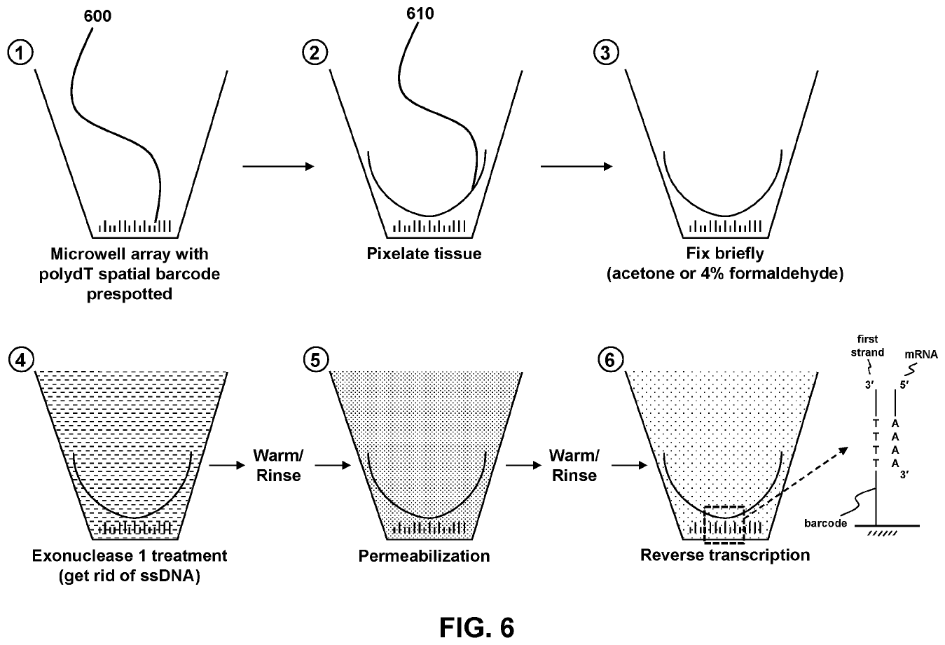

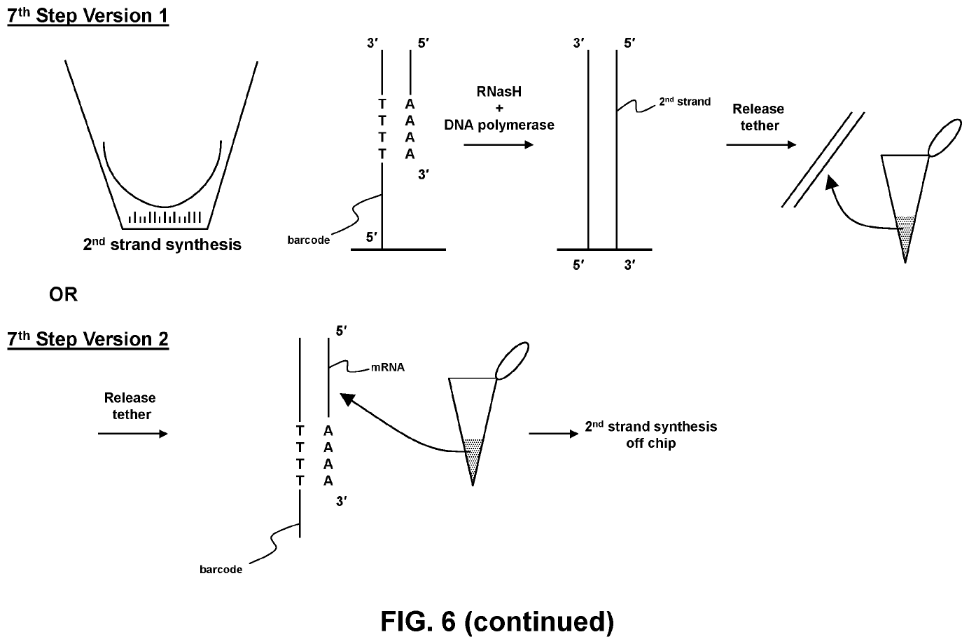

FIG. 6 is a schematic diagram of steps that may be used for a spatial transcriptomics application using any of the microarrays provided herein, including microarrays made by any of the methods described herein. In step 1, a microwell array, including of the instant invention, is provided with polyT spatial barcodes 600. As desired, the barcodes can be photocleavable. In step 2, tissue 610 is pixelated. The tissue may be fixed, as summarized in step 3, such as via acetone or formaldehyde. The fixation may help preserve RNA for processing at room temperature, including by inactivating RNases. In step 4, ssDNA may be removed, including via an exonuclease treatment. This removes unwanted priming of mRNA during a reverse transcriptase (RT) step. See, e.g., Stahl et al. “Visualization and analysis of gene expression in tissue sections by spatial transcriptomics” Science 353(6294): 78-82 (Jul. 1, 2016). A wash/rinse is performed and in step 5 the samples in the wells are permeabilized. Various permeabilization methods are available, including: (i) Proteinase K—5-10 μg/mL for 15-30 minutes at 45° C.; or (ii) 0.1% pepsin in 0.1 M HCl for 10 minutes at 37° C. See, e.g., Stahl et al. A wash/rinse is performed and in step 6, reverse transcription occurs against any mRNA sequences bound to the barcode, including polyT-containing barcodes. In this step, the well may have various reagents necessary for the polymerase reaction, including a first strand buffer, 5 mM DTT, 500 μM dNTP each, 0.19 μg/μL BSA (bovine serum albumin), 50 ng/μL actinomycin, 1% DMSO, 20 U/μL superscript Ill, 2 U/μL RnaseOUT (RNase h). See e.g., Stahl et al. Another option is using a commercially-available kit, such as Ambion Arrayscript™ RT/kit (ThermoFisher Scientific).

In the seventh step of FIG. 6, two versions are provided for second strand synthesis. RNase H and DNA polymerase may provide the second strand synthesis as illustrated in the top panels labeled “Version 1”. The double strand may be released. Alternatively the second stand may be synthesized off chip (bottom panel). Relevant reagents for the second strand synthesis include second strand buffer, dNTP, BSA, DNA polymerase, RNase H. 5 μL T4 DNA polymerase may be added later. See, e.g., Stahl et al., Ambion's kit or other kits (www.neb.com/products/e7760-nebnext-ultra-ii-directional-rna-library-prep-kit-for-illumina#product%20information).

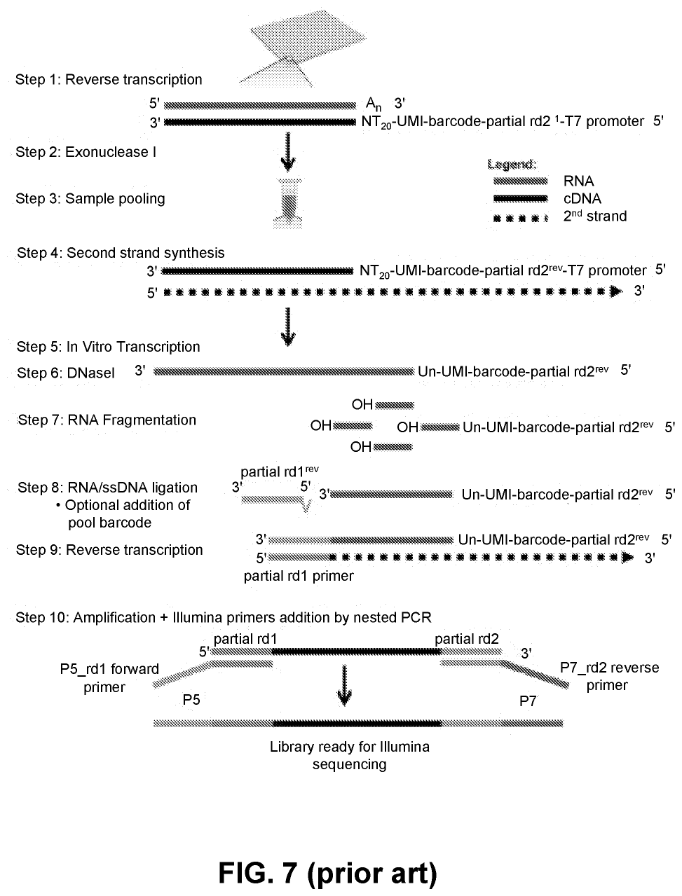

Various off-chip steps are available, including: step 8—In vitro transcription to linearly amplify RNA using in vitro transcription; and step 9 (RNA fragmentation and ligate 3′ adapter (illumina) (see other steps in schematic of FIG. 7). FIG. 7 summarizes schematically various steps, from Figure S2 of Jaitin et al. “Massively parallel single cell RNA-Seq for marker-free decomposition of tissues into cell types” Science 343(6172): 776-779 (Feb. 14, 2014), to convert single-cell RNA samples to sequencing-ready DNA libraries, including tagging, pooling, amplification and fragmentation. Other studies follow this protocol.

### Example 4: Barcoding Strategies

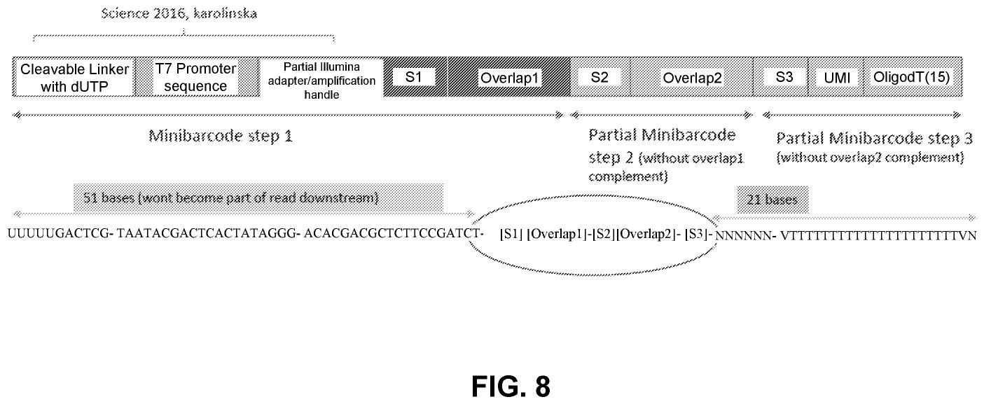

FIG. 8 is a barcode summary schematic, including useful specifications and probability calculations. The barcode is illustrated as having MBC1, MBC2 and MBC3 components at the top panel and corresponding sequence information in the bottom panel. In one scenario, 100 unique MBC's are added for each on-chip addition step, for 3 on-chip addition steps (n=3). In this manner, a total of 100³ barcodes are possible after the third bead addition. For the 100 unique MBC (e.g., one per each of the 100 bead “populations”), S1=xxxx (4 base length) (e.g., 4⁴ or 256 unique combinations possible for the four nucleotides, out of which this scenario uses 100). S2=xxxx. S3=xxxx; wherein x is A, T, C or G. For an overlap of 10 bases long, the total length is S1+overlap1+S2+overlap2+S3=32 bases, with a total barcode sequenced of 32+21=53 bases only. For an overlap of 20 bases long, then S1+overlap1+S2+overlap2+S3=52 bases, with a total barcode sequenced=52+21=73 bases only (see, e.g, Klein et al. “Droplet barcoding for single-cell transcriptomics applied to embryonic stem cells.” Cell 161: 1187-1201 (May 21, 2015).

Based on the 100 unique MBC and 3 additions, 100³ or 1,000,000 unique barcodes are available. A microarray having 1000×1000 array of 7 μm well with 3 μm spacing (as one example) provides 1,000,000 wells with a spatial footprint of 1 cm×1 cm. Accordingly, large tissue samples can be processed. The probability of having 2 sequences identical is 1×10⁻⁶. The sequencing specs may include paired end reads (Illumina) and can sequence about 2500 cells with 200,000 to 1,000,000 reads per cell (roughly 1 million to 2 million reads per cell are suitable to adequately sequences cellular transcript, see, e.g., Grun et al. “Design and analysis of single-cell sequencing experiments” Cell 163(4): 799-810 (Nov. 5, 2015).

General design rules include, for S1 equal representation of each of the 4 nucleotides (A, T, C, G). For S1, S2 and S3, any two data sets out of XXX must be different by at least two bases, leaving 243 possible combinations, out of which we select 100. Ov1, Ov2 equal 22 bases, and we may refer to Weitz Cell paper (2015) with a post T7 promoter sequence:

(SEQ ID NO: 1)
ATACCACCATGGCTCTTTCCCTACACGACGCTCTTCCGATCT,
wherein:
(SEQ ID NO: 2)
ACACGACGCTCTTCCGATCT
corresponds to StahI (Science 2016-amplification
handle/Illumina partial-length = 20)
(SEQ ID NO: 3)
CTCTTTCCCTACACGACGCTCTTCCGATCT
mentioned as illumine adapter partial in Weitz-
length = 30
(SEQ ID NO: 4)
ATACCACCATGG-
spacer, 12 bases.

Cleaving sequences may be as described in Stahl paper (Science 2016). Ligation adapters may include those described in Weitz, Stahl and Jaitin publications.

Accordingly, a barcode may be generally described as:

U cleaver-T7-adapter-S1 (100/243 with rules as described above)-Ov1-S2 (100/243 with rules as described above)-ov2-S3 (100/243 with rules described as above)-UMI-polyT (about 20 bases T, 10 bases UMI).

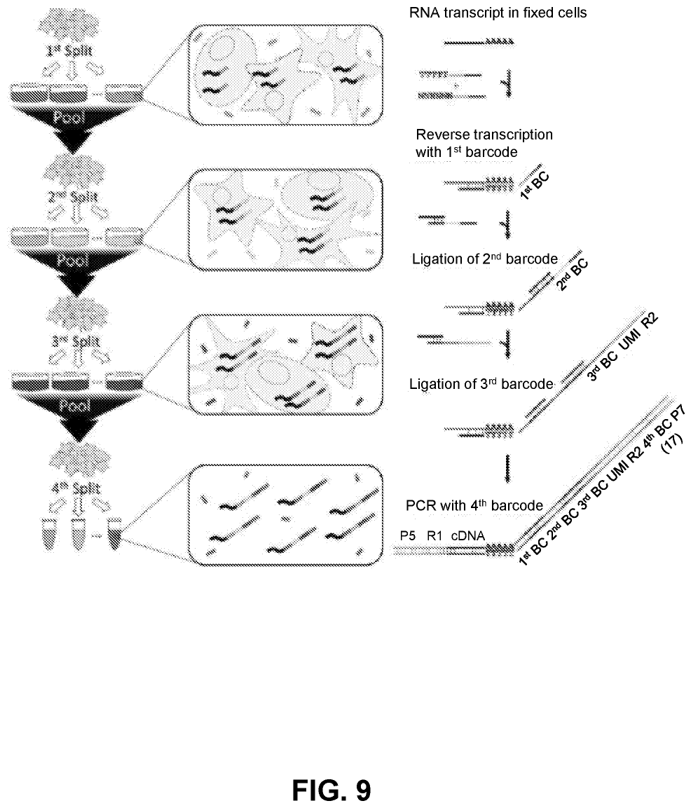

FIG. 9 is adapted from Rosenberg et al. “Single-cell profiling of the developing mouse brain and spinal cord with split-pool barcoding” Science 360(6385): 176-182 (Apr. 13, 2018) and is adapted to the instant process, except on beads where the MBC's are ligated together.

The process may use LNA (locked nucleic acids) as one of the bases in the overlap region in the MBC's. For example, template switching oligo in smart-seq2 protocol uses a similar approach by incorporating 1 LNA base in the oligo to increase Tm by 1 to 10° C. See, e.g., Picelli et al. “Full-length RNA-seq from single cells using Smart-seq2” Nat. Protoc. 9(1): 171-181 (January 2014).

The methods are compatible with pre-enrichment, such as in vitro transcription or PCR based enrichment. In isolated wells in the microarrays described herein, we improve the capture of low abundance transcripts by removing/reducing amplification bias and improving enrichment efficiency. See, e.g., Gole et al. “Massively parallel polymerase cloning and genome sequencing of single cells using nanoliter microwells” Nat. Biotech. 31: 1126-1132 (2013) (MIDS in microwell array (whole genome amplification).

Microfluidics may incorporate any of a variety of inlets (carrier fluid inlet, reagent inlet), filters, channels, including droplet stabilization channel, and collection outlets. See, e.g., Cell (2015).

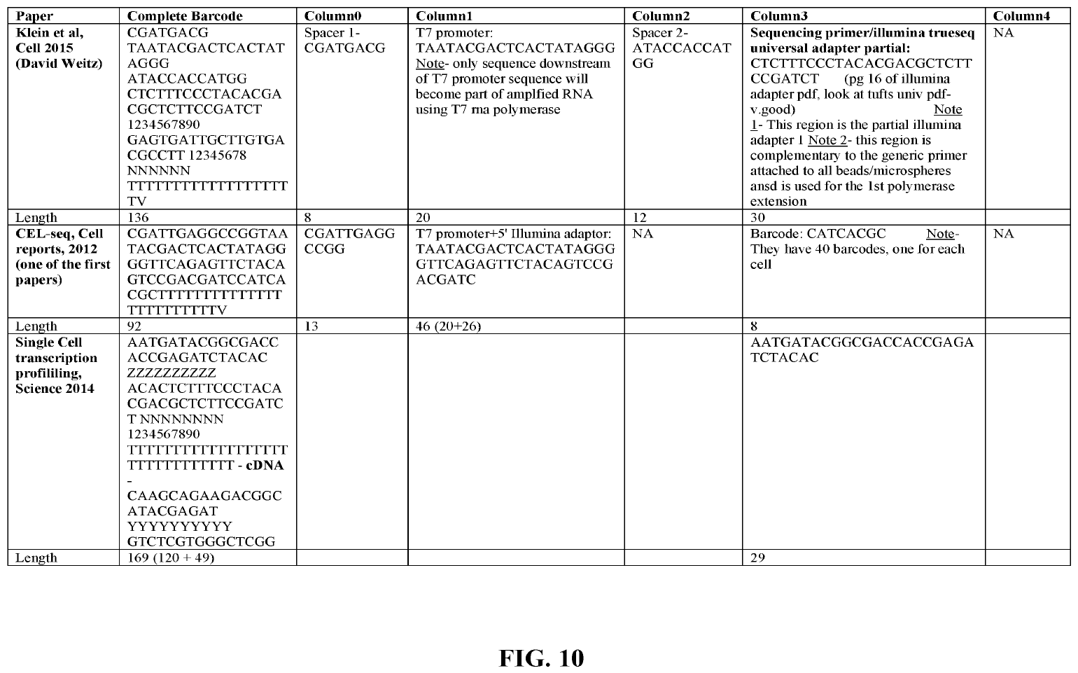

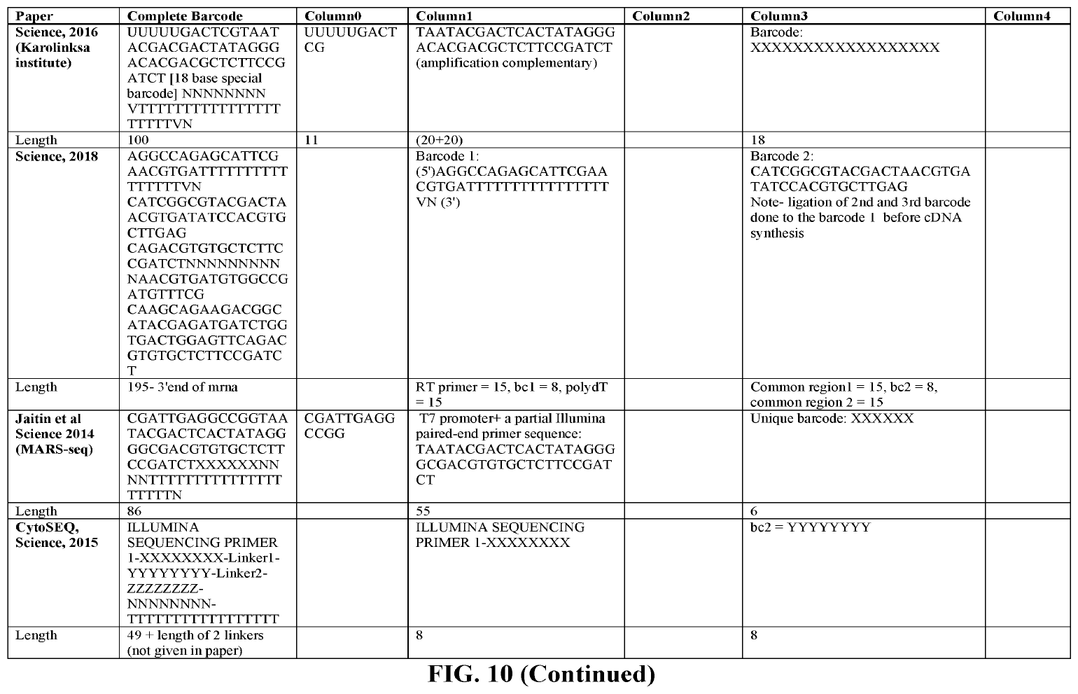

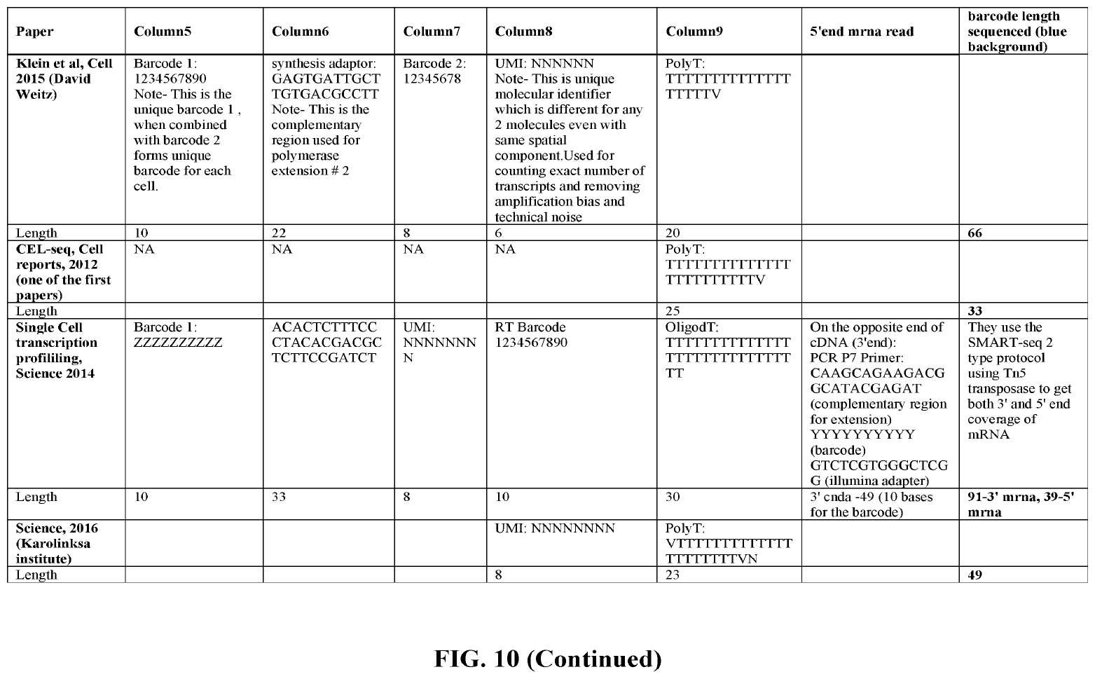

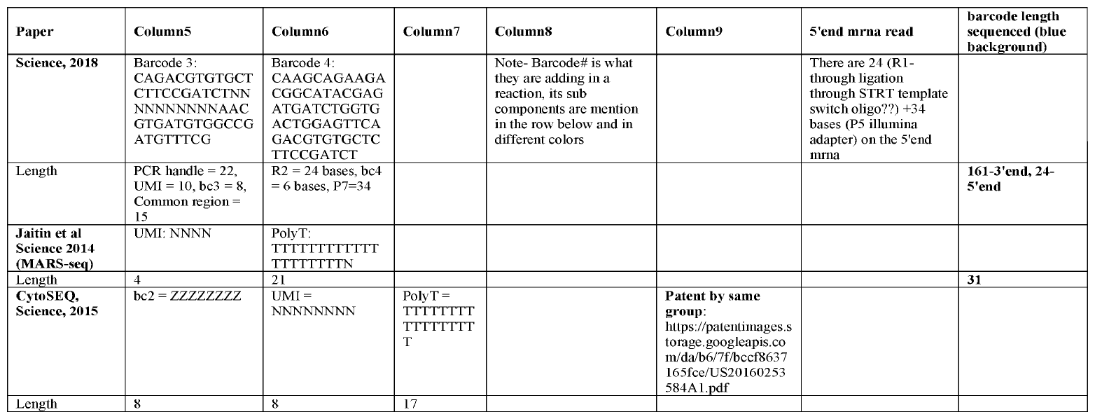

Design rules for spatial barcodes may incorporate various barcoding strategies in RNA-seq, as summarized in FIG. 10 (see also US Pat. Pub. 2016/0253584). The following rules provide guidance on spatial barcode design:
1. Have a unique molecular identifier (UMI) to remove amplification bias and technical noise. UMI sequence is different for each molecule (even between two barcodes with same spatial sequence).
2. For 3′ end sequencing, only 1 barcode per transcript required. use oligodT barcoding strategy.
3. Total barcode length can be 150-160 bases for paired end reads, such as using Illumina platform. Total read length in such cases become 150*2=300 bases. Read 1 gives the barcode sequence, and read 2 gives the transcript sequence.
4. When using in vitro transcription for enrichment, the usual barcode for the RT step is: polyT-UMI-unique barcode-5′illumina adapter-T7 promotor sequence (3′-5′ direction). T7 promoter is sued for in vitro transcription; 5′ illumine adapter is for illumine library preparation; UMI is to count the exact number of transcripts; unique barcode provides spatial or single cell info; polyT is to hybridize the 3′ end of polyA mRNA. See, e.g., Grun et al. Cell 163(4): 799-810 (Nov. 5, 2015) and Wu et al. “Single-cell transcriptional analysis” Ann. Rev. Anal. Chem. 10: 439-462 (June 2017).
5. T7 promoter sequence, 5′ illumine adapter—not read in the final sequence read step (e.g., not counted in the 150 base read length sequenced).
6. For the unique barcode portion immediately following the adapter, the barcodes are designed in groups of four, such that the first five nucleotides have equal representation of all four nucleotides to allow for template generation and crosstalk corrections that are based on the first four nucleotides read in the Illumina platform. See, e.g., Hashimshony et al. “CEL-Seq: Single-Cell RNA-Seq by Multiplexed Linear Amplification.” Cell Reports 2(3): 666-673 (Sep. 27, 2012).
7. Any two barcodes preferentially differ by at least two bases to minimize mis-assignments.

Exemplary mini-barcode sequences include, but are not limited to:

### Example—Minibarcode 1: Fixed

(SEQ ID NO: 5)
UUUUUGACTCGTAATACGACTCACTATAGGGACACGACGCTCTTCCG
ATCT.
Variable-Positional:
ATGCAA
Fixed-overlap1:
(SEQ ID NO: 6)
ACA TTG TGT CCA GGT GC.
Minibarcode 2: Fixed-overlap1 complement:
(SEQ ID NO: 7)
1 TGT AAC ACA GGT CCA CG.
Variable-Positional (S2c):
TTACG
Fixed-overlap2 complement:
(SEQ ID NO: 8)
AGT CTC CAT ACG CAC TCG.
Minibarcode 3
Fixed-overlap2 complement:
(SEQ ID NO: 9)
1 AGT CTC CAT ACG CAC TCG.
Variable-Positional (S3c = S2c):
TTACG
UMI-anchored oligodT complement:
(SEQ ID NO: 10)
NNNNNN-BAAAAAAAAAAAAAAAAAAAAABN.

### STATEMENTS REGARDING INCORPORATION BY REFERENCE AND VARIATIONS

All references throughout this application, for example patent documents including issued or granted patents or equivalents; patent application publications; and non-patent literature documents or other source material; are hereby incorporated by reference herein in their entireties, as though individually incorporated by reference, to the extent each reference is at least partially not inconsistent with the disclosure in this application (for example, a reference that is partially inconsistent is incorporated by reference except for the partially inconsistent portion of the reference).

The terms and expressions which have been employed herein are used as terms of description and not of limitation, and there is no intention in the use of such terms and expressions of excluding any equivalents of the features shown and described or portions thereof, but it is recognized that various modifications are possible within the scope of the invention claimed. Thus, it should be understood that although the present invention has been specifically disclosed by preferred embodiments, exemplary embodiments and optional features, modification and variation of the concepts herein disclosed may be resorted to by those skilled in the art, and that such modifications and variations are considered to be within the scope of this invention as defined by the appended claims. The specific embodiments provided herein are examples of useful embodiments of the present invention and it will be apparent to one skilled in the art that the present invention may be carried out using a large number of variations of the devices, device components, methods steps set forth in the present description. As will be obvious to one of skill in the art, methods and devices useful for the present methods can include a large number of optional composition and processing elements and steps.

As used herein and in the appended claims, the singular forms “a”, “an”, and “the” include plural reference unless the context clearly dictates otherwise. Thus, for example, reference to “a cell” includes a plurality of such cells and equivalents thereof known to those skilled in the art. As well, the terms “a” (or “an”), “one or more” and “at least one” can be used interchangeably herein. It is also to be noted that the terms “comprising”, “including”, and “having” can be used interchangeably. The expression “of any of claims XX-YY” (wherein XX and YY refer to claim numbers) is intended to provide a multiple dependent claim in the alternative form, and in some embodiments is interchangeable with the expression “as in any one of claims XX-YY.”

When a group of substituents is disclosed herein, it is understood that all individual members of that group and all subgroups, are disclosed separately. When a Markush group or other grouping is used herein, all individual members of the group and all combinations and subcombinations possible of the group are intended to be individually included in the disclosure.

Every device, system, formulation, combination of components, or method described or exemplified herein can be used to practice the invention, unless otherwise stated.

Whenever a range is given in the specification, for example, a temperature range, a time range, or a composition or concentration range, all intermediate ranges and subranges, as well as all individual values included in the ranges given are intended to be included in the disclosure. It will be understood that any subranges or individual values in a range or subrange that are included in the description herein can be excluded from the claims herein.

All patents and publications mentioned in the specification are indicative of the levels of skill of those skilled in the art to which the invention pertains. References cited herein are incorporated by reference herein in their entirety to indicate the state of the art as of their publication or filing date and it is intended that this information can be employed herein, if needed, to exclude specific embodiments that are in the prior art. For example, when composition of matter are claimed, it should be understood that compounds known and available in the art prior to Applicant's invention, including compounds for which an enabling disclosure is provided in the references cited herein, are not intended to be included in the composition of matter claims herein.

As used herein, “comprising” is synonymous with “including,” “containing,” or “characterized by,” and is inclusive or open-ended and does not exclude additional, unrecited elements or method steps. As used herein, “consisting of” excludes any element, step, or ingredient not specified in the claim element. As used herein, “consisting essentially of” does not exclude materials or steps that do not materially affect the basic and novel characteristics of the claim. In each instance herein any of the terms “comprising”, “consisting essentially of” and “consisting of” may be replaced with either of the other two terms. The invention illustratively described herein suitably may be practiced in the absence of any element or elements, limitation or limitations which is not specifically disclosed herein.

One of ordinary skill in the art will appreciate that starting materials, biological materials, reagents, synthetic methods, purification methods, analytical methods, assay methods, and biological methods other than those specifically exemplified can be employed in the practice of the invention without resort to undue experimentation. All art-known functional equivalents, of any such materials and methods are intended to be included in this invention. The terms and expressions which have been employed are used as terms of description and not of limitation, and there is no intention that in the use of such terms and expressions of excluding any equivalents of the features shown and described or portions thereof, but it is recognized that various modifications are possible within the scope of the invention claimed. Thus, it should be understood that although the present invention has been specifically disclosed by preferred embodiments and optional features, modification and variation of the concepts herein disclosed may be resorted to by those skilled in the art, and that such modifications and variations are considered to be within the scope of this invention as defined by the appended claims.

Table of sequence listings:

| SEQ | | |
|---|---|---|
| ID | | |
| NO: | Description | Sequence |
| 1 | Weitz | ATACCACCATGGCTCTTTCCCTACAC |
| | | GACGCTCTTCCGATCT |
| 2 | Amp handle | ACACGACGCTCTTCCGATCT |
| | of SEQ ID | |
| | NO: 1 | |
| 3 | Adapter | CTCTTTCCCTACACGACGCTCTTCCG |
| | partial of | ATCT |
| | SEQ ID NO: 1 | |
| 4 | Spacer of | ATACCACCATGG |
| | SEQ ID NO: 1 | |
| 5 | MBC1 Fixed | UUUUUGACTCGTAATACGACTCACTA |
| | | TAGGGACACGACGCTCTTCCGATCT |
| 6 | MBC1 Fixed- | ACA TTG TGT CCA GGT GC |
| | overlap 1 | |
| 7 | MBC2 Fixed- | TGT AAC ACA GGT CCA CG |
| | overlap 1 | |
| | complement | |
| 8 | MBC2 Fixed- | AGT CTC CAT ACG CAC TCG |
| | overlap 2 | |
| | complement | |
| 9 | MBC3 Fixed- | AGT CTC CAT ACG CAC TCG |
| | overlap2 | |
| | complement | |
| 10 | UMI-anchored | NNNNNN-BAAAAAAAAAAAAAAAAAA |
| | oligodT | AAABN |
| | complement | |

1. A method of making a spatially-barcoded microarray, the method comprising the steps of:
a) providing distinguishable mini-barcoded beads, wherein the distinguishable mini-barcoded beads comprise a plurality of distinct bead populations, with each bead member of a distinct population having an identical mini-barcode sequence;
b) simultaneously delivering the plurality of distinguishable barcoded beads to a plurality of wells of a microarray, wherein a single bead is provided to each well;
c) imaging the plurality of wells to identify the population type of each bead in each of the plurality of wells and thereby identify the mini-barcode (MBC) in each well;
d) removing the mini-barcodes from the beads and connecting the mini-barcode or a polymerase product of the mini-barcode to a surface of the well in which the bead is located; and
e) removing the beads from the wells;
thereby making a spatially-barcoded microarray.

2. The method of claim 1, further comprising: sequentially repeating steps a)-c) and, in step d) removing the mini-barcodes from the beads and ligating and/or performing polymerase extension of the mini-barcode and/or a polymerase product of the mini-barcode to a previously introduced mini-barcode in each of the plurality of wells, thereby providing a spatial pattern of barcode sequences over the plurality of wells; each barcode sequence comprising a plurality of known mini-barcode sequences and/or polymerase products from a plurality of mini-barcodes.

3. The method of claim 2, wherein the sequentially repeating steps is repeated multiple times, including between two and 10 times.

4. The method of claim 1, wherein a bead in a well is optically, electrically, or mechanically identifiable by population type.

5. The method of claim 4, wherein the distinct bead populations are identifiable by a bead color and/or a fluorescent label connected to the beads, the method further comprising the step of optically imaging the beads in the wells.

6. The method of claim 1, wherein the step of simultaneously delivering the plurality of identifiable barcoded beads to the plurality of wells comprises mixing the beads in a liquid and fluidically delivering the beads in the liquid to the plurality of wells.

7. The method of claim 1, wherein the step of simultaneously delivering the plurality of identifiable barcoded beads to the plurality of wells comprises applying a physical force to insert the beads into the wells, including a centrifugal force, an electrical force, and/or a magnetic force.

8. The method of claim 1, wherein the plurality of bead populations are formed from one population or more than one distinct populations, wherein the at least one bead-delivering step comprises a plurality of sub-delivery steps with each sub-delivery step comprising bead delivery to only a fraction of the plurality of the wells; wherein there are optionally at least 5 distinct bead populations.

9. The method of claim 1, further comprising applying the plurality of bead populations to a fraction of the total number of wells of the microarray, and repeating the applying steps in one or more additional application steps to fill all wells of the microarray with a single bead, thereby effectively increasing the number of bead populations with unique mini-barcodes applied to the microarray.

10. The method of claim 1, wherein the imaging step comprises optically or electrically analyzing the microarray to identify the population type of each single bead in each well.

11. The method of claim 1, wherein the removing the mini-barcode step comprises cleaving the mini-barcode from the bead surface at a cleavage site.

12. The method of claim 1, wherein the connecting comprises ligating the mini-barcode to the well surface or to a mini-barcode previously connected to the well surface.

13. The method of claim 1, wherein the connecting comprises making an amplicon in the well by a polymerase reaction involving the mini-barcode and connecting the amplicon from the polymerase reaction to the well surface or to a previously connected mini-barcode, the method further comprising the step of delivering reagents to the wells to perform the polymerase reaction in the well.

14. The method of claim 1, wherein the barcode connected to the well surface comprises a plurality of mini-barcodes, the barcode having a nucleotide length that is greater than or equal to 80 bases and less than or equal to 150 bases.

15. The method of claim 1, wherein the mini-barcode has a sequence length of between 20 and 70 bases.

16. The method of claim 1, wherein the bead and/or the well has a diameter selected so that only one bead occupies the well volume; wherein the bead diameter is greater than or equal to 200 nm and less than 5 mm.

17. (canceled)

18. The method of claim 1, wherein the wells have a well density that is greater than or equal to 0.008 wells/μm² (8,000 wells/mm²) or a well spacing distance between adjacent wells that is less than or equal to 10 μm.

19. The method of claim 18, having a spatial resolution that is sub-cellular for intracellular characterization of a biological cell.

20. The method claim 1, wherein the wells of the microarray have an average diameter that is greater than 200 nm and less than or equal to 5 mm, and optionally the ratio of bead diameter to well diameter is greater than 0.5 and optionally the ratio of bead diameter to well height is greater than 0.5.

21. (canceled)

22. A high-density microarray comprising:
a microarray including a plurality of wells, each well having a known barcode sequence to form a spatially patterned array of nucleotide sequence barcodes;
wherein the plurality of wells have a spatial density that is greater than or equal to 8,000 wells/mm² and a spatial resolution configured for intracellular or intercellular characterization of biological cells from a biological material.

23. (canceled)

24. (canceled)

25. The high-density microarray of claim 22, wherein the wells have a side wall configured to pixelate a biological tissue for spatial analysis of the biological tissue.

26. (canceled)

27. A method of sequencing a transcriptome from a biological material, the method comprising the steps of:
providing the high density microarray of claim 22;
overlaying the microarray with the biological material;
pixelating the biological material into the plurality of wells;
performing cDNA synthesis on the biological material in the plurality of wells, wherein non-barcoded random hexamers act as primers for the cDNA synthesis;
ligating the barcoded sequences connected to the well surfaces that are complementary to the synthesized cDNA; and
performing RNA sequencing on each of the plurality of wells.

28. The method of claim 26, further comprising a pre-amplification step to amplify rare nucleic acid targets.

29. The method of claim 27, for sequencing a transcriptome at a sub-cellular spatial resolution.

30. The method of claim 27, wherein the sequenced transcriptome is a complete transcriptome of a tissue or a cell.

31. (canceled)

32. The method of claim 27, further comprising the step of delivering to the microwells microbeads having one or more reagents connected thereto.

33. The method of claim 32, wherein the reagents comprise ligase enzymes and buffers lyophilized on a surface of the bead.

34. The method of claim 32, wherein the delivering step comprises magnetic and/or electrical pulldown of beads that are magnetic and/or electrically charged, and/or centrifugation of beads dispersed in a carrier liquid.

35. The method of claim 27, further comprising the step of providing reagents dispersed in a liquid to the wells of the microarray.

36. The method of claim 27, wherein after the performing cDNA synthesis, the synthesized cDNA is dried in the microwells and a mixture of ligation and amplification reagents are provided to the microwells.

37. The method of claim 27, wherein lyophilized amplification reagents and enzymes are delivered to the wells of the microarray after the ligation step.

38. (canceled)

39. The method of claim 27, wherein a single biological cell spans a plurality of wells, optionally between 4 and 10 adjacent wells.

40. The method of claim 27, wherein the spatial density of the wells are selected to provide a spatial resolution that is better than or equal to 20 μm.


SEQUENCE LISTING (10 sequences)

SEQ ID NO: 1 - DNA, 42 bp, Artificial Sequence
  Synthetic Construct
ataccaccatggctctttccctacacgacgctcttccgatct

SEQ ID NO: 2 - DNA, 20 bp, Artificial Sequence
  Synthetic Construct
acacgacgctcttccgatct

SEQ ID NO: 3 - DNA, 30 bp, Artificial Sequence
  Synthetic Construct
ctctttccctacacgacgctcttccgatct

SEQ ID NO: 4 - DNA, 12 bp, Artificial Sequence
  Synthetic Construct
ataccaccatgg

SEQ ID NO: 5 - DNA, 51 bp, Artificial Sequence
  Synthetic Construct
uuuuugactcgtaatacgactcactatagggacacgacgctcttccgatct

SEQ ID NO: 6 - DNA, 17 bp, Artificial Sequence
  Synthetic Construct
acattgtgtccaggtgc

SEQ ID NO: 7 - DNA, 17 bp, Artificial Sequence
  Synthetic Construct
tgtaacacaggtccacg

SEQ ID NO: 8 - DNA, 18 bp, Artificial Sequence
  Synthetic Construct
agtctccatacgcactcg

SEQ ID NO: 9 - DNA, 18 bp, Artificial Sequence
  Synthetic Construct
agtctccatacgcactcg

SEQ ID NO: 10 - protein, 21 aa, Artificial Sequence
  Synthetic Construct
AAAAAAAAAAAAAAAAAAAAA